(19) United States
(12) Patent Application Publication
(10) Pub. No.: US 2024/0027353 A1
(43) Pub. Date: Jan. 25, 2024

(54) OPTICAL MEASUREMENT METHOD, OPTICAL MEASUREMENT SYSTEM, AND TEST KIT

(71) Applicants: CANON KABUSHIKI KAISHA, Tokyo (JP); Canon Medical Systems Corporation, Otawara-shi (JP)

(72) Inventors: Minako NAKASU, Tokyo (JP); Daisuke SASAGURI, Yokohama (JP); Norishige KAKEGAWA, Tokyo (JP); Satoshi YAMABI, Yokohama (JP); Seiko YOSHIMURA, Nasushiobara (JP)

(73) Assignees: CANON KABUSHIKI KAISHA, Tokyo (JP); Canon Medical Systems Corporation, Otawara-shi (JP)

(21) Appl. No.: 18/357,573

(22) Filed: Jul. 24, 2023

(30) Foreign Application Priority Data
Jul. 25, 2022 (JP) 2022-118166

(51) Int. Cl. G01N 21/77; G01N 33/543

(52) U.S. Cl. CPC G01N 21/7703; G01N 33/54333

(57) ABSTRACT

An optical measurement method comprises a holding step, a measurement step, and a first application step. In the holding step, a specimen solution containing a specimen and the magnetic particles is held in a specific magnetic field state, in which a magnetic field not lower than a first magnetic field for moving magnetic particles closer to a substrate is not applied, on the substrate or in a storage container different from the substrate. In the measurement step, an elapsed time from a start reference time of the specific magnetic field state is measured. In the first application step, after the elapsed time reaches a predetermined time, the first magnetic field is applied, for a first application time, by a magnet, to the specimen solution introduced to the substrate.

### CROSS-REFERENCE TO RELATED APPLICATION(S)

This application is based upon and claims the benefit of priority from prior Japanese Patent Application No. 2022-118166, filed Jul. 25, 2022, the entire contents of which are incorporated herein by reference.

### FIELD

Embodiments described herein relate generally to an optical measurement method, an optical measurement system, and a test kit.

### BACKGROUND

In the biosensor field, there is provided a measurement method of in vitro diagnosis for optically measuring, by an optical measurement system using a translucent substrate, a measurement target substance such as an antigen contained in a specimen, and determining whether the specimen contains the measurement target substance. Presently, it has been required to develop a high sensitive measurement method in order to determine the presence of a smaller amount of the measurement target substance.

This optical measurement method uses a mechanism of immobilizing the first substance, that specifically reacts with the measurement target substance, on the detection surface of the translucent substrate, and bonding the first substance and the measurement target substance. As one method of improving sensitivity, it is effective to promote the reaction of the first substance with the measurement target substance.

As a technique aiming at improving sensitivity, there is provided a method of operating, as a stirrer, magnetic particles to which the second substance that specifically reacts with the measurement target substance is bonded. That is, by repeating spontaneous sedimentation of magnetic particles and pull-back of the magnetic particles in the upper direction of the substrate by applying a magnetic field, the magnetic particles are largely moved to stir a specimen solution. This diffuses the measurement target substance in the specimen solution to promote the reaction of the magnetic particles of the second substance with the measurement target substance, and it is thus expected to obtain higher detection sensitivity within a shorter time. Then, presently, it has been required to further improve sensitivity to a smaller amount of the measurement target substance.

### BRIEF DESCRIPTION OF THE DRAWINGS

FIG. 1 is a block diagram showing an example of the configuration of an optical measurement system according to the first embodiment.

FIG. 2 is a view showing the outer appearance of a test kit according to the first embodiment.

FIG. 3 is a view showing the optical measurement principle of an optical measurement apparatus.

FIG. 4 is a view showing the sectional structure of the first magnetic particle.

FIG. 5 is a view showing the sectional structure of the second magnetic particle.

FIG. 6 is a flowchart illustrating the general processing procedure of optical measurement by the optical measurement system according to the first embodiment.

FIG. 7 is a flowchart illustrating the processing procedure of optical measurement executed by the optical measurement apparatus in step S604 of FIG. 6.

FIG. 8 is a timing chart showing a time-series change of the signal strength of a light detection signal according to the first embodiment.

FIG. 9 is a view showing the behavior of magnetic particles in a reaction tank by comparing a specific magnetic field state (zero magnetic field state) and a state of applying a lower magnetic field.

FIG. 10 is a block diagram showing an example of the configuration of an optical measurement system according to the second embodiment.

FIG. 11 is a view showing the outer appearance of a test kit according to the second embodiment.

FIG. 12 is a flowchart illustrating the general processing procedure of optical measurement by the optical measurement system according to the second embodiment.

FIG. 13 is a flowchart illustrating the processing procedure of optical measurement executed by an optical measurement apparatus in step S1208 of FIG. 12.

FIG. 14 is a timing chart showing a time-series change of the signal strength of a light detection signal according to the second embodiment.

FIG. 15 is a graph showing a measurement result according to Example 2.

### DETAILED DESCRIPTION

An optical measurement method according to one embodiment is an optical measurement method using an optical measurement system including a translucent substrate on which a first substance capable of being specifically bonded to a measurement target substance is immobilized, a magnet configured to apply a magnetic field for moving magnetic particles to which a second substance capable of being specifically bonded to the measurement target substance is bonded, and an optical device configured to cause light to enter the substrate and detect light having propagated through the substrate and exited from the substrate. The optical measurement method according to the embodiment comprises a holding step, a measurement step, and a first application step. In the holding step, a specimen solution containing a specimen and the magnetic particles is held in a specific magnetic field state, in which a magnetic field not lower than a first magnetic field for moving the magnetic particles closer to the substrate is not applied, on the substrate or in a storage container different from the substrate. In the measurement step, an elapsed time from a start reference time of the specific magnetic field state is measured. In the first application step, after the elapsed time reaches a predetermined time, the first magnetic field is applied, for a first application time, by the magnet, to the specimen solution introduced to the substrate.

Embodiments of an optical measurement method, an optical measurement system, and a test kit will be described in detail below with reference to the accompanying drawings.

### First Embodiment

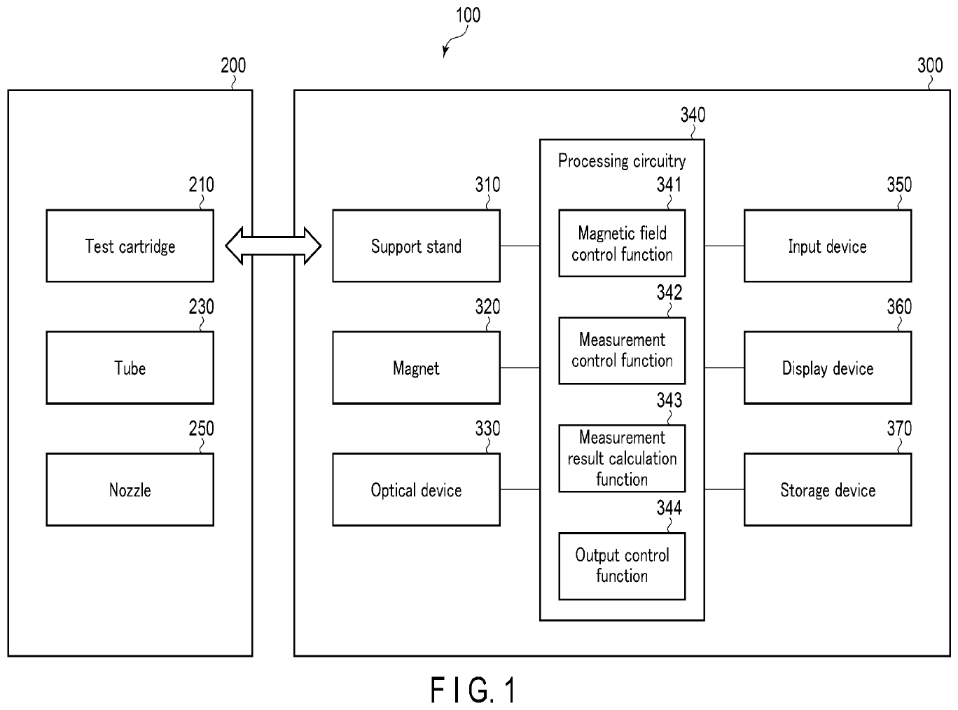

FIG. 1 is a block diagram showing an example of the configuration of an optical measurement system 100 according to the first embodiment. The optical measurement system 100 is a system that optically measures a measurement target substance. As long as the measurement target substance is a substance detectable by the optical measurement system 100, it is not specifically limited. Examples of the measurement target substance are antigens such as influenza virus, adenovirus, RS (Respiratory Syncytial) virus, and coronavirus (COVID-19 and the like). As shown in FIG. 1, the optical measurement system 100 includes a test kit 200 and an optical measurement apparatus 300.

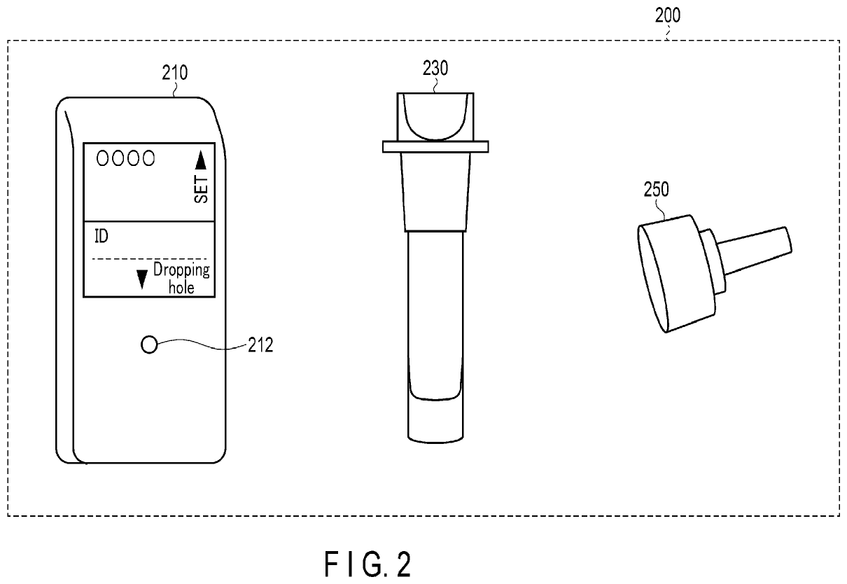

FIG. 2 is a view showing the outer appearance of the test kit 200 according to the first embodiment. As shown in FIGS. 1 and 2, the test kit 200 includes an test cartridge 210, a tube 230, and a nozzle 250. The test cartridge 210 includes a substrate (to be referred to as a translucent substrate hereinafter) which is translucent and on which the first substance that is specifically bonded to the measurement target substance is immobilized. The test cartridge 210 is provided with a dropping hole 212 connecting to an internally provided reaction tank. The test cartridge 210 is attached to the optical measurement apparatus 300. The tube 230 is a container containing a specimen treatment solution. As an example, a buffer solution containing a surfactant is used as the specimen treatment solution. A specimen is suspended in the specimen treatment solution. The nozzle 250 is a nozzle attached to the tube 230. Magnetic particles to which the second substance that is specifically bonded to the measurement target substance is bonded adhere to the nozzle 250. A mixed solution of the specimen treatment solution, the measurement target substance, and the magnetic particles is dropped into the dropping hole 212 of the test cartridge 210. The mixed solution of the specimen treatment solution, the measurement target substance, and the magnetic particles will be referred to as a test solution hereinafter.

As shown in FIG. 1, the optical measurement apparatus 300 includes a support stand 310, a magnet 320, an optical device 330, a processing circuitry 340, an input device 350, a display device 360, and a storage device 370. The support stand 310, the magnet 320, the optical device 330, the processing circuitry 340, the input device 350, the display device 360, and the storage device 370 are connected via a signal line such as a bus so as to be able to transmit/receive signals.

The support stand 310 is a support mechanism for detachably supporting the test cartridge 210. Attachment/detachment of the test cartridge 210 to/from the support stand 310 is detected electrically, magnetically, or mechanically.

The magnet 320 applies a magnetic field for moving magnetic particles introduced to the test cartridge 210. The magnetic field applied by the magnet 320 is controlled by a magnetic field control function 341 of the processing circuitry 340. The magnet 320 is an example of a magnetic field application unit.

The optical device 330 emits light to the translucent substrate of the test cartridge 210, and detects light having propagated through the translucent substrate and exited from the translucent substrate. An electrical signal (to be referred to as a light detection signal hereinafter) representing the intensity of the detected light is supplied to the processing circuitry 340. The optical device 330 is an example of an optical unit.

The processing circuitry 340 is a processor that functions as a control center of the optical measurement apparatus 300. By executing programs stored in the storage device 370 and the like, the processing circuitry 340 implements functions corresponding to the programs, that is, the magnetic field control function 341, a measurement control function 342, a measurement result calculation function 343, and an output control function 344. Note that this embodiment will describe a case where the magnetic field control function 341, the measurement control function 342, the measurement result calculation function 343, and the output control function 344 are implemented by the single physical processor but the present invention is not limited to this. For example, a plurality of independent processors may be combined to form a processing circuitry, and the field control function 341, the measurement control function 342, the measurement result calculation function 343, and the output control function 344 may be implemented when the respective processors execute the programs, respectively.

By the magnetic field control function 341, the processing circuitry 340 controls the timing of applying the magnetic field by the magnet 320. More specifically, the applied magnetic field includes a magnetic field (to be referred to as a lower magnetic field hereinafter) for moving the magnetic particles closer to the translucent substrate and a magnetic field (to be referred to as an upper magnetic field hereinafter) for moving the magnetic particles away from the translucent substrate. The processing circuitry 340 controls the timing of applying the lower magnetic field, the timing of applying the upper magnetic field, the application intensity of each magnetic field, an application time, and the like. The magnetic field control function 341 is an example of a magnetic field application unit.

By the measurement control function 342, the processing circuitry 340 controls optical measurement by the optical device 330. As an example, the processing circuitry 340 controls the timing of turning on and off light irradiation by the optical device 330. The measurement control function 342 is an example of an optical control unit.

By the measurement result calculation function 343, the processing circuitry 340 calculates the measurement result of optical measurement concerning the measurement target substance with respect to the test solution based on the light detection signal from the optical device 330. More specifically, as the measurement result, various index values based on the strength of the light detection signal and a determination result concerning negative/positive of the measurement target substance based on the index values and the like can be calculated. The measurement result calculation function 343 is an example of a calculation unit.

By the output control function 344, the processing circuitry 340 outputs various kinds of information via an output interface such as the display device 360. As an example, the processing circuitry 340 displays, on the display device 360, the various index values and the determination result calculated by the measurement result calculation function 343.

The input device 350 accepts various input operations from an operator, and converts the accepted input operations into operation signals. The operations signals are supplied to the processing circuitry 340. As an example, a physical switch, a touch panel, a touch pad, a joystick, a keyboard, or the like can be used as the input device 350. A voice input device that recognizes utterance of the operator sensed by a microphone and converts it into an operation signal may be used as the input device 350.

The display device 360 displays various kinds of information by the output control function 344. As an example, a liquid crystal display (LCD), a CRT (Cathode Ray Tube) display, an organic EL display (OELD: Organic Electro Luminescence Display), a plasma display, or another arbitrary display can appropriately be used as the display device 360. Furthermore, the display device 360 may be a projector.

The storage device 370 is a storage device such as a ROM (Read Only Memory), a RAM (Random Access Memory), an HDD (Hard Disk Drive), an SSD (Solid State Drive), or an integrated circuit storage device that stores various kinds of information. Furthermore, the storage device 370 may be a driving device that reads/writes various kinds information from/in a portable storage medium such as a flash memory, a CD-ROM, or a DVD. Note that the storage device 370 need not always be implemented by one storage device. For example, the storage device 370 may be implemented by a plurality of storage devices. The storage device 370 stores one or more programs according to this embodiment. For example, the program may be stored in advance in the storage device 370. Alternatively, for example, the program may be stored in a non-transitory storage medium and distributed, and then read out from the non-transitory storage medium and installed on the storage device 370. Furthermore, a control program may be, for example, downloaded from a network and installed on the storage device 370.

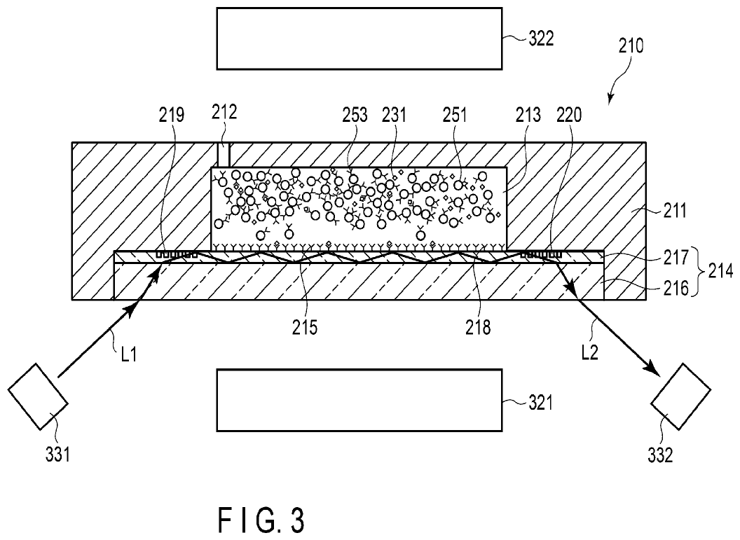

FIG. 3 is a view showing the optical measurement principle of the optical measurement apparatus 300. FIG. 3 shows the section of the optical measurement apparatus 300 in which the test cartridge 210 is attached to the support stand 310 (not shown in FIG. 3).

The test cartridge 210 includes a housing 211. The housing 211 is made of a resin such as ABS (acrylonitrile butadiene styrene), and formed in an almost rectangular parallelepiped shape. The housing 211 may be colored with black for the purpose of blocking light. The dropping hole 212 is formed in the upper surface of the housing 211. The dropping hole 212 connects to a reaction tank 213 in the housing 211 via a channel.

A translucent substrate 214 is provided on the rear surface of the housing 211. The translucent substrate 214 is a substrate which is translucent and on which a first substance 215 that is specifically bonded to a measurement target substance 231 is immobilized. More specifically, the translucent substrate 214 includes a translucent base portion 216. The base portion 216 is made of, for example, alkali-free glass. An optical waveguide 217 is formed on the upper surface of the base portion 216.

As an example, a planar optical waveguide can be used as the optical waveguide 217. The optical waveguide 217 can be made of, for example, a thermosetting resin such as phenol resin, epoxy resin, or acrylic resin, a photo-curable resin, or alkali-free glass. The optical waveguide 217 has transmittance with respect to predetermined light, and is preferably made of, for example, a resin with a refractive index higher than that of the base portion 216.

Part of the upper surface of the optical waveguide 217 forms a detection surface (sensing area) 218, and serves as the bottom surface of the reaction tank 213. The first substance 215 that specifically reacts with the measurement target substance 231 is immobilized on the detection surface 218. The first substance 215 is immobilized by, for example, a hydrophobic interaction or chemical bond to the upper surface of the optical waveguide 217. The detection surface 218 indicates a region where near-field light (evanescent light) can be generated on the upper surface of the optical waveguide 217.

The test solution is introduced to the reaction tank 213 via the dropping hole 212. As described above, a second substance 253 that is specifically bonded to the measurement target substance is bonded to magnetic particles 251. Furthermore, the first substance 215 that specifically reacts with the measurement target substance 231 is immobilized on the detection surface 218. If the measurement target substance 231 is an antigen, an antibody (secondary antibody) is used for the second substance 253, and the antibody (secondary antibody) is used for the first substance 215.

Note that the combination of the measurement target substance, the first substance 215, and the second substance 253 is not limited to the above-described combination of the antigen and antibody. For example, a combination of sugar and lectin, a combination of a nucleotide chain and its complementary nucleotide chain, a combination of ligand and a receptor, or the like may be used.

A grating 219 for incidence and a grating 220 for reflection are provided in two edge portions of the upper surface of the base portion 216, respectively. The grating 219 is configured to reflect (diffract) light, and is arranged at a position where light is made to enter the optical waveguide 217. The grating 220 is configured to reflect (diffract) light, and is arranged at a position where light propagating through the optical waveguide 217 is reflected to the outside.

As described above, the optical device 330 is provided in the optical measurement apparatus 300. As shown in FIG. 3, more specifically, the optical device 330 includes a light source 331 and a light detector 332. The light source 331 emits a light beam L1 toward the translucent substrate 214 under the control of the processing circuitry 340. As an example, a red laser diode is used as the light source 331 but a laser diode of another color or a light emitting diode may be used. The light beam L1 may be shaped to be almost parallel by an additionally added lens or the like. The light detector 332 detects a light beam L2 exiting from the translucent substrate 214. A photodiode is used as the light detector 332. The light detector 332 generates a light detection signal representing the intensity of the detected light beam L2.

As shown in FIG. 3, the magnet 320 is provided in the optical measurement apparatus 300. As shown in FIG. 3, the magnet 320 includes a lower magnetic field magnet 321 and an upper magnetic field magnet 322 to sandwich the test cartridge 210. Each of the lower magnetic field magnet 321 and the upper magnetic field magnet 322 is implemented by a permanent magnetic or an electromagnet. The lower magnetic field magnet 321 applies a magnetic field (lower magnetic field) for moving the magnetic particles 251 closer to the detection surface 218 under the control of the processing circuitry 340. The upper magnetic field magnet 322 applies a magnetic field (upper magnetic field) for moving the magnetic particles 251 away from the detection surface 218 under the control of the processing circuitry 340.

The structure of the magnetic particle that can be used in the first embodiment will be described next.

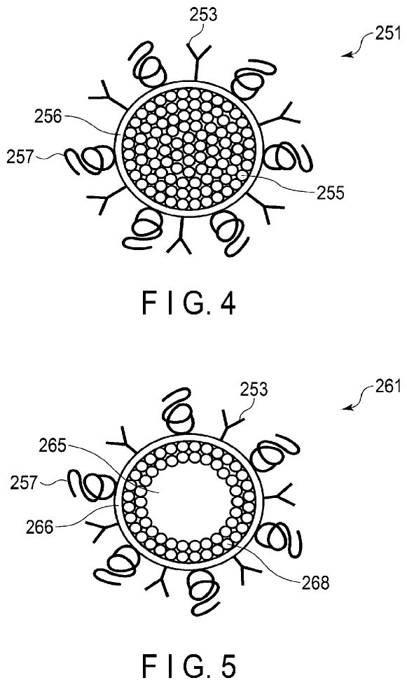

FIG. 4 is a view showing the sectional structure of the first magnetic particle 251. As shown in FIG. 4, the magnetic particle 251 includes a core 255 formed from an aggregate of magnetic materials (nanoparticles). The core 255 is covered with a hydrophilic layer 256. The hydrophilic layer 256 is made of, for example, glycidyl methacrylate. Furthermore, the second substance 253 and blocking polymer 257 are bonded to the hydrophilic layer 256.

FIG. 5 is a view showing the sectional structure of a second magnetic particle 261. As shown in FIG. 5, the second magnetic particle 261 includes a polymer core 265. As a polymer material, for example, polystyrene is used. The core 265 is covered with a magnetic layer 268. The magnetic layer 268 is covered with a hydrophilic layer 266. The second substance 253 and the blocking polymer 257 are bonded to the hydrophilic layer 266.

As will be apparent by comparing FIGS. 4 and 5, since the first magnetic particle 251 includes the core 255 made of the magnetic material, magnetic field responsiveness is high and the time taken for the first magnetic particle 251 to reach the detection surface 218 at the time of applying the lower magnetic field is short, as compared with the second magnetic particle 261 whose core 265 is not made of the magnetic material.

Next, the processing procedure of optical measurement by the optical measurement system 100 according to the first embodiment will be described. In the following description of the first embodiment, assume that the measurement target substance is an antigen, and each of the first substance and the second substance is an antibody. Assume also that as the material particle, the first magnetic particle whose core is made of a magnetic material is used.

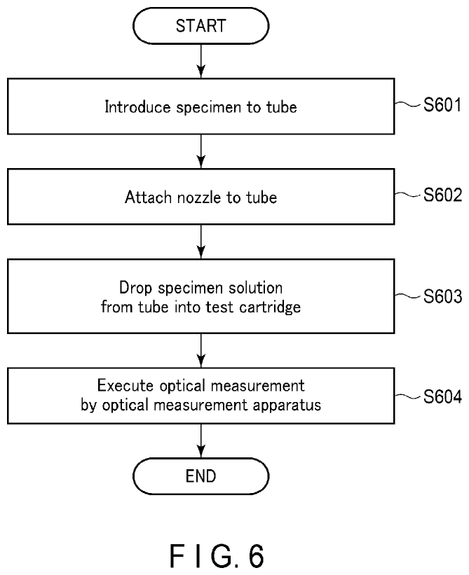

FIG. 6 is a flowchart illustrating the general processing procedure of optical measurement by the optical measurement system 100 according to the first embodiment. As shown in FIG. 6, an operator first introduces a specimen to the tube 230 (step S601). The type of the specimen and a specimen collection method according to the first embodiment are not specifically limited. As the type of the specimen, nasal mucus, saliva, sputum, blood, lymph, or any other substance that can be collected from a human body can be used. As an example, if the specimen is a nasal swab, a nasal swab is collected by wiping the nasopharynx with a sterile cotton applicator (swab), the sterile cotton applicator is dipped in the specimen treatment solution in the tube 230, and the specimen treatment solution is stirred. Thus, the specimen is suspended in the specimen treatment solution.

After step S601 is performed, the operator attaches the nozzle 250 to the tube 230 (step S602). The first magnetic particles to which the second substance that is specifically bonded to the measurement target substance is bonded adhere to the nozzle 250. More specifically, a filter is provided inside the nozzle 250, and the magnetic particles are immobilized on the filter.

After step S602 is performed, the operator drops the specimen solution from the tube 230 into the test cartridge 210 (step S603). Note that before step S603, the operator has attached the test cartridge 210 to the support stand 310 of the optical measurement apparatus 300. The operator drops the specimen solution into the dropping hole 212 of the test cartridge 210 attached to the support stand 310. At this time, if the suspended solution in the tube 230 passes through the filter of the nozzle 250, the first magnetic particles immobilized on the filter are eluted to the suspended solution. This introduces the specimen solution as a mixed solution of the specimen, the specimen treatment solution, and the first magnetic particles to the reaction tank 213 of the test cartridge 210.

After step S603 is performed, the optical measurement apparatus 300 executes optical measurement for the specimen (step S604). The optical measurement apparatus 300 executes optical measurement by receiving an optical measurement start trigger. The optical measurement start trigger can arbitrarily be set. As an example, the closing of the cover of the optical measurement apparatus 300, a lapse of a preset time after the cover is closed, or the pressing of a measurement start button by the operator is set as the trigger.

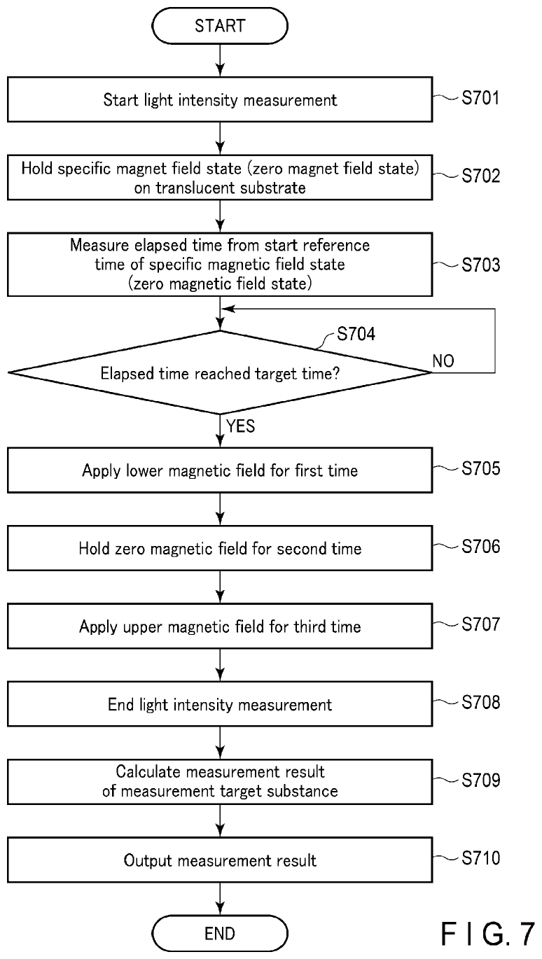

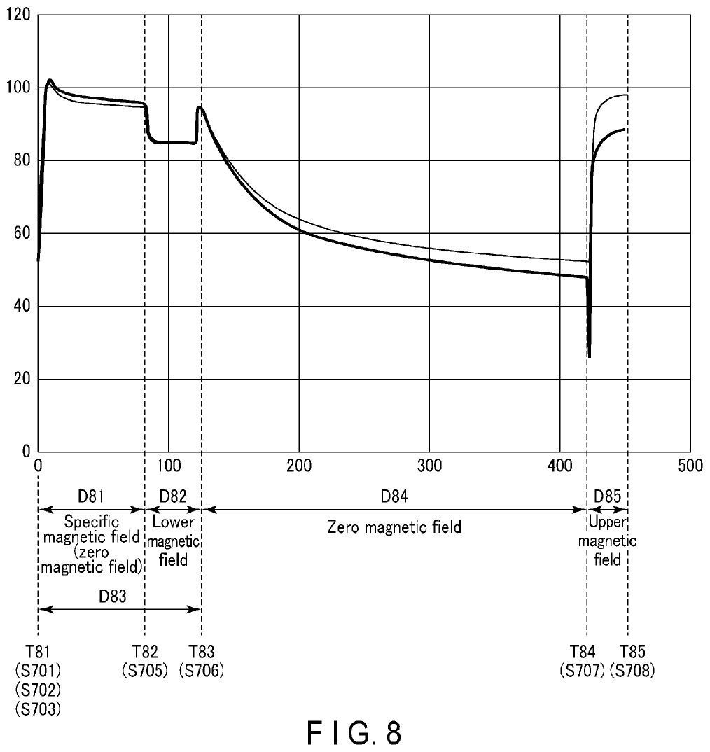

FIG. 7 is a flowchart illustrating the processing procedure of optical measurement executed by the optical measurement apparatus 300 in step S604 of FIG. 6. FIG. 8 is a timing chart showing a time-series change of the signal strength of the light detection signal according to the first embodiment. In the timing chart shown in FIG. 8, the ordinate represents the signal strength [AU] of the light detection signal, and the abscissa represents the time [sec]. In FIG. 8, a thick line represents the time-series signal strength of the specimen containing the measurement target substance, and a thin line represents the time-series signal strength of the specimen containing no measurement target substance.

As shown in FIG. 7, the processing circuitry 340 of the optical measurement apparatus 300 starts light intensity measurement by the measurement control function 342 (step S701). Start time T81 of light intensity measurement corresponds to the time when the optical measurement start trigger such as the closing of the cover of the optical measurement apparatus 300, a lapse of the preset time after the cover is closed, or the pressing of the measurement start button by the operator is received. In step S701, the processing circuitry 340 controls the light source 331 to cause a light beam to enter the translucent substrate 214. The light beam that has entered the translucent substrate 214 is reflected or diffracted by the grating 219 via the base portion 216, enters the optical waveguide 217, and propagates through the optical waveguide 217. Then, the light beam is reflected or diffracted by the grating 220, and exits from the translucent substrate 214. The light detector 332 detects the exited light beam, and generates a light detection signal representing the intensity of the detected light beam. The light detection signal is supplied to the processing circuitry 340.

If the reaction tank 213 is empty, the light propagating through the optical waveguide 217 is not totally reflected, thereby generating evanescent light (leakage light) on the detection surface 218. In this case, the signal strength of the light detection signal has a low value, as compared with a case where the light is totally reflected. After the time elapses after step S701, the test solution dropped into the test cartridge 210 starts to flow into the reaction tank 213. If the detection surface 218 is dipped in the test solution, the light beam propagating through the optical waveguide 217 is totally reflected. That is, as the time elapses after step S701, the signal strength of the light detection signal increases.

After step S701 is performed, the processing circuitry 340 holds the specific magnetic field state by the magnetic field control function 341 (step S702). In step S702, the processing circuitry 340 controls the lower magnetic field magnet 321 and the upper magnetic field magnet 322 to hold the test solution in the specific magnetic field in the reaction tank 213. The specific magnetic field state indicates a state in which a magnetic field with intensity equal to or higher than that of the lower magnetic field to be applied in step S705 is not applied. Typically, the specific magnetic field state indicates a zero magnetic field state in which no magnetic field is applied. For example, the processing circuitry 340 forms the zero magnetic field state by stopping the application of the magnetic field from each of the lower magnetic field magnet 321 and the upper magnetic field magnet 322. Note that zero magnetic field state need only be a state in which the net intensity of the magnetic field existing in the reaction tank 213 is almost zero, and includes, for example, a state in which the intensity of the composite magnetic field of the lower magnetic field from the lower magnetic field magnet 321 and the upper magnetic field from the upper magnetic field magnet 322 is almost zero in the reaction tank 213. In step S702, the processing circuitry 340 holds the test solution in the zero magnetic field state in the reaction tank 213.

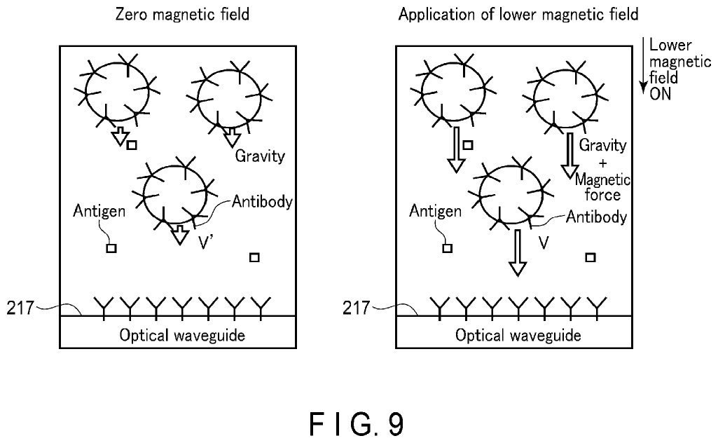

FIG. 9 is a view showing the behavior of the magnetic particles in the reaction tank 213 by comparing the specific magnetic field state (zero magnetic field state) and the state of applying the lower magnetic field. As shown in FIG. 9, the reaction tank is filled with the test solution. The first magnetic particles and the antigen (measurement target substance) are suspended in the test solution. The antibody (second substance) is bonded to the first magnetic particles. The bottom surface of the reaction tank is the detection surface 218 of the optical waveguide, and the antibody (first substance) is immobilized on the detection surface 218. In the zero magnetic field, the first magnetic particles freely fall by the gravity. On the other hand, in the state of applying the lower magnetic field, the first magnetic particles fall by the resultant force of the gravity and the lower magnetic field. That is, a velocity v′ of the first magnetic particles that freely fall is lower than a velocity v of the first magnetic particles in the state of applying the lower magnetic field. Therefore, as compared with the state of applying the lower magnetic field, in the zero magnetic field state, since it is possible to sufficiently ensure the time during which the antigen reacts with the antibody, it can be said that the antigen readily reacts with the antibody. That is, it can be said that sensitivity is high in the zero magnetic field state, as compared with the state of applying the lower magnetic field.

In the zero magnetic field state, the first magnetic particles spontaneously precipitate, and reach the detection surface 218 at a low velocity. In a case where the first magnetic particles reach the detection surface 218, the light propagating through the optical waveguide 217 leaks from the detection surface 218 due to the first magnetic particles. Therefore, in the zero magnetic field state, the signal strength of the light detection signal gradually decreases.

After step S702 is performed, the processing circuitry 340 measures the elapsed time from the start reference time of the specific magnetic field state (zero magnetic field state) by the magnetic field control function 341 (step S703). The start reference time can arbitrarily be set. As an example, the time at which the processing circuitry 340 receives the optical measurement start trigger is set as the start reference time. More specifically, the start reference time is set by the closing of the cover of the optical measurement apparatus 300, a lapse of the preset time after the cover is closed, or the pressing of the measurement start button by the operator. Steps S701, S702, and S703 are executed at almost same time T81. The processing circuitry 340 measures the elapsed time using a hardware timer or a software timer. This manages the duration of the zero magnetic field state.

After step S703 is performed, the processing circuitry 340 determines whether the elapsed time has reached a target time (to be referred to as a standstill time) D81, by the magnetic field control function 341 (step S704). The standby time D81 corresponds to a time during which the specific magnetic field state (zero magnetic field state) is maintained. The standby time D81 can be set to an arbitrary value in accordance with the purpose. The standby time D81 is qualitatively a time taken to bond the first magnetic particles to the antigen via the antibody, and is set to a time during which a sufficient amount of the first magnetic particles can be bonded to the antigen.

If it is determined that the elapsed time has reached the standby time D81 (YES in step S704) (T82), the processing circuitry 340 applies the lower magnetic field for a first time D82 by the magnetic field control function 341 (step S705). In step S705, the processing circuitry 340 controls the lower magnetic field magnet 321 to apply the lower magnetic field for the first time D82. By applying the lower magnetic field, the first magnetic particles are attracted to the detection surface 218. As compared with the second magnetic particles (polymer cores), the first magnetic particles have high magnetic field responsiveness and the time taken for the first magnetic particles to reach the detection surface 218 is short. Since the first magnetic particles are attracted to the detection surface 218 one after another, bundles of the first magnetic particles are arranged on the detection surface 218 in a square shape along the magnetic line of force of the lower magnetic field, and thus a number of bundles are arrayed on the detection surface 218.

The first time D82 as the lower magnetic field application time is set to an arbitrary time. The first time D82 is qualitatively set to a time taken for a sufficient amount of the first magnetic particles that can exist in the reaction tank 213 to reach the detection surface 218.

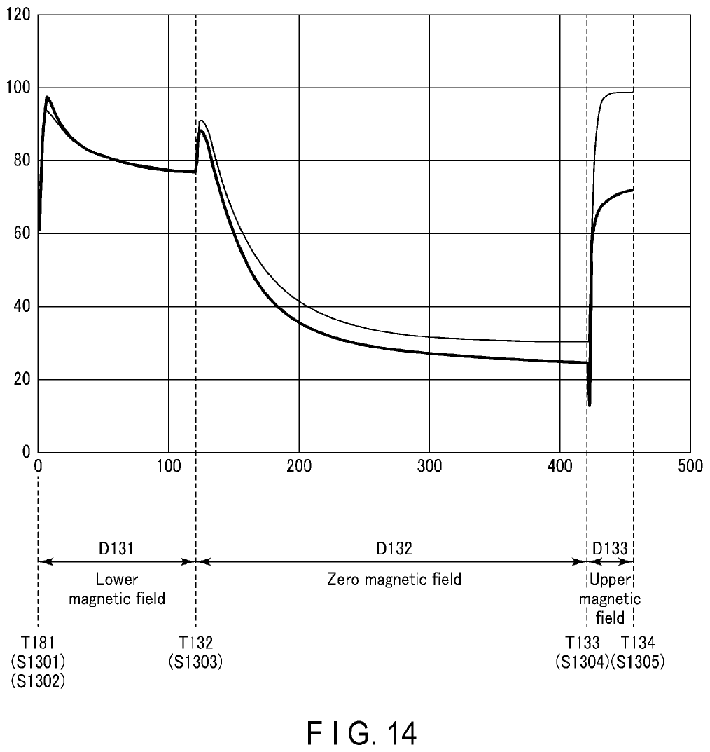

The setting of the standby time D81 as the duration of the specific magnetic field state will now be described in detail. If the second magnetic particles are used, the lower magnetic field is immediately applied from measurement start reference time T81 without providing the specific magnetic field state (see FIG. 14). The lower magnetic field application time (to be referred to as the second lower magnetic field application time hereinafter) when using the second magnetic particles is set based on the time taken for the second magnetic particles to reach the detection surface 218 at the time of applying the lower magnetic field.

As describe above, as compared with magnetic particles according to a comparative example, the magnetic particles according to the first embodiment have a high falling velocity at the time of applying the lower magnetic field. The lower magnetic field application time (first time) D82 when using the first magnetic particles according to the first embodiment can be short, as compared with the second lower magnetic field application time. Assume that a total time D83 of the standby time D81 and the first time D82 is set to the second lower magnetic field application time. The first time D82 is set based on the time taken for the first magnetic particles to reach the detection surface 218. A practical value of the first time D82 is decided based on an experience value obtained based on an experiment or simulation. The standby time D81 is set based on a value obtained by subtracting the first time D82 from the total time D83. The first time D82 can largely be shortened, as compared with the second lower magnetic field application time. For example, if the second lower magnetic field application time, in other words, the total time D83 is 120 sec, and the first time D82 is 40 sec, the standby time D81 is set to 80 sec (=120 sec-40 sec). The standby time D81 can be set to a large value, as compared with the first time D82. This can ensure the time for causing the magnetic particles to react with the antigen as long as possible under the restriction of the total time D83.

After step S705 is performed (T83), the processing circuitry 340 holds the zero magnetic field for a second time D84 by the magnetic field control function 341 (step S706). In step S706, the processing circuitry 340 stops the application of the lower magnetic field by the lower magnetic field magnet 321. By stopping the application of the lower magnetic field, the bundles of the first magnetic particles that are arranged on the detection surface 218 in a square shape are loosened to reach the detection surface 218. At this time, the intensity of leakage light from the detection surface 218 becomes maximum, and the signal strength of the light detection signal becomes minimum accordingly.

After step S706 is performed (T84), the processing circuitry 340 applies the upper magnetic field for a third time D85 by the magnetic field control function 341 (step S707). In step S707, the processing circuitry 340 applies the upper magnetic field from the upper magnetic field magnet 322 for the third time D85. By applying the upper magnetic field, the first magnetic particles having reached the detection surface 218 are separated from the detection surface 218. Along with this, the signal strength of the light detection signal increases. On the other hand, since the first magnetic particles bonded to the antigen are bonded to the antibody immobilized on the detection surface 218, they remain without being separated from the detection surface 218 even by applying the upper magnetic field. Therefore, if the specimen contains no antigen, the signal strength of the light detection signal returns to the initial state. However, if the specimen contains the antigen, the signal strength of the light detection signal remains at a value lower than in the initial state without returning to the initial state.

After step S707 is performed (T85), the processing circuitry 340 ends the light intensity measurement by the measurement control function 342 (step S708). In step S708, the processing circuitry 340 stops radiation with the light beam from the light source 331.

After step S708 is performed, the processing circuitry 340 calculates the measurement result of the measurement target substance by the measurement result calculation function 343 (step S709). In step S709, the processing circuitry 340 calculates a sensitivity value and a variation rate integrated value based on the time-series signal intensity of the light detection signal supplied from the light detector 332. The sensitivity value represents the degree of attenuation of the signal strength after a specific time elapses from the measurement start reference time. As another example, a sensitivity value S is defined by equation (1) below. Note that I(200s) represents the signal strength 200 sec after the measurement start reference time, and I(899s) represents the signal strength 899 sec after the measurement start reference time.

S[%]=100-1(899s)/I(200s)*80 (1)

The variation rate integrated value represents the integrated value of the variation rate of the signal strength for the specific period. The specific period corresponds to the calculation target period of the variation rate integrated value. As an example, the specific period is set to a period of 213 sec to 233 sec after the measurement start reference time. A moving average value is calculated based on the signal strength for every sec during the specific period. Furthermore, a moving average reference value is calculated. The moving average reference value represents the average value of three time points (for example, 108 s, 109 s, and 110 s) within the measurement time. The time points are set in accordance with an inspection item. The variation rate is defined by equation (2) below. The variation rate is calculated for every sec during the specific period, and the variation rate is integrated for the specific period, thereby calculating the variation rate integrated value.

variation rate=moving average value/moving average reference value*10000−10000 (2)

The processing circuitry 340 determines a positive or negative result based on comparison between the sensitivity value and a determination threshold. More specifically, if the sensitivity value exceeds the determination threshold, the positive result is determined. If the sensitivity value is lower than the determination threshold, the negative result is determined. The determination threshold of the sensitivity value is set to an arbitrary value based on experiences. The processing circuitry 340 determines a positive or negative result based on comparison between the variation rate integrated value and a determination threshold. More specifically, if the variation rate integrated value exceeds the determination threshold, the positive result is determined. The determination threshold of the variation rate integrated value is set to an arbitrary value based on experiences.

After step S709 is performed, the processing circuitry 340 outputs the measurement result calculated in step S709 by the output control function 344 (step S710). More specifically, the processing circuitry 340 displays, on the display device 360, the sensitivity value, the variation rate integrated value, the determination result based on the sensitivity value, and the determination result based on the variation rate integrated value in an arbitrary layout.

Then, the optical measurement by the optical measurement apparatus 300 ends.

According to the above-described first embodiment, after the zero magnetic field state is held for a given time before the application of the lower magnetic field, it becomes possible to apply the lower magnetic field. By holding the zero magnetic field state for a given time, it is possible to move the magnetic particles at a low velocity, as compared with the state of applying the lower magnetic field. This can sufficiently ensure a time during which the antibody bonded to the magnetic particles reacts with the antigen, thereby improving the reaction efficiency of the antigen-antibody reaction, and also improving the sensitivity characteristic.

Note that as another method of promoting the antigen-antibody reaction, there is a method of alternately applying the upper magnetic field and the lower magnetic field to stir the magnetic particles before the application of the lower magnetic field in step S705. By stirring the magnetic particles, an opportunity for the magnetic particles contacting the antigen is increased. However, the magnetic particles are not always bonded to the antigen. In addition, the magnetic particles and the antigen may be dissociated from each other due to a physical action. According to the method of holding the zero magnetic field state as in the first embodiment, it is possible to suppress a situation in which the magnetic particles and the antigen are dissociated from each other due to a physical action, and it is thus expected to improve the sensitivity characteristic, as compared with the method of stirring.

Note that the specific magnetic field state in step S702 is assumed to be the zero magnetic field state. However, this embodiment is not limited to this. In the specific magnetic field state, the magnetic field may be applied as long as the moving velocity of the magnetic particles is low, or the time taken for the magnetic particles to reach the detection surface 218 is long, as compared with the lower magnetic field (to be referred to as the main lower magnetic field hereinafter) applied in step S705. As an example, in step S702, by the magnetic field control function 341, the processing circuitry 340 may control the lower magnetic field magnet 321 to apply a lower magnetic field (to be referred to as a weak lower magnetic field hereinafter) with a strength lower than that of the main lower magnetic field. As another example, by the magnetic field control function 341, the processing circuitry 340 may control the upper magnetic field magnet 322 to apply an upper magnetic field (to be referred to as a weak upper magnetic field hereinafter) with such low strength that the magnetic particles move toward the detection surface 218. That is, the strength of the weak upper magnetic field is lower than that of the upper magnetic field (to be referred to as the main upper magnetic field hereinafter) applied in step S707. As still another example, by the magnetic field control function 341, the processing circuitry 340 may time-divisionally apply a weak lower magnetic field with the multistage strength lower than the strength of the main lower magnetic field, or time-divisionally apply the weak lower magnetic field, the weak upper magnetic field, and the zero magnetic field.

### Example 1

Optical measurement was executed by the following procedure using the optical measurement system 100 according to the first embodiment. An influenza virus kit “Rapiim Flu-AB” (Canon Medical Systems Corporation) was used as the test kit 200, and a protein analysis apparatus “Rapiim Eye 10” (Canon Medical Systems Corporation) was used as the optical measurement apparatus 300. The influenza virus kit “Rapiim Flu-AB” is formed from an influenza virus test cartridge, a nozzle, and a tube.

<Method>

0. Setting of Measurement Sequence

A measurement sequence of turning off a magnetic field for 80 sec after measurement starts, applying a lower magnetic field for 40 sec from 81st sec to 120th sec, turning off the magnetic field for 300 sec from 121st sec to 420th sec, and applying an upper magnetic field for 30 sec from 421st sec to 450th sec was newly set in the apparatus main body (Rapiim Eye 10) (the total measurement time was 450 sec).

On the other hand, as the standard sequence of a current product, a lower magnetic field was applied from 1st sec after measurement starts to 120th sec, the magnetic field was turned off for 300 sec from 121st sec to 420th sec, and an upper magnetic field was applied for 30 sec from 421st sec to 450th sec (the total measurement time was 450 sec).

1. Preparation of Test Solution

After dipping a swab in a specimen mixed with an influenza B antigen, the swab was dipped in a treatment solution in the tube, and was pulled out from the tube while squeezing the specimen. After that, the nozzle was attached to the tube.

2. Setting of Test Cartridge

The influenza virus test cartridge was attached to the apparatus main body (Rapiim Eye 10).

3. Measurement

A predetermined amount of the test solution prepared in 1 was dropped into the cartridge set in 2, and the slide cover of the apparatus main body was closed, thereby starting measurement. The measurement ended 7 minutes and 30 seconds later.

<Result>

In a case where the newly set measurement sequence was executed, a positive result was determined with respect to influenza B virus. The sensitivity value was 24%. On the other hand, in a case where the standard sequence of the current product was executed as well, a positive result was determined with respect to influenza B virus. The sensitivity value was 20%. By holding the specimen treatment solution in the zero magnetic field state for a given time before the application of the lower magnetic field, it was possible to improve the sensitivity (second embodiment).

In the above-described first embodiment, a specimen solution containing a specimen and magnetic particles is held in the specific magnetic field state on the translucent substrate 214 of the optical measurement apparatus 300. The second embodiment assumes that a specimen solution is held in a specific magnetic field state in a storage container different from a translucent substrate 214. An optical measurement system according to the second embodiment will be described below. Note that the same reference numerals as in the first embodiment denote components having almost the same functions in the following description, and a repetitive description will be provided, as needed.

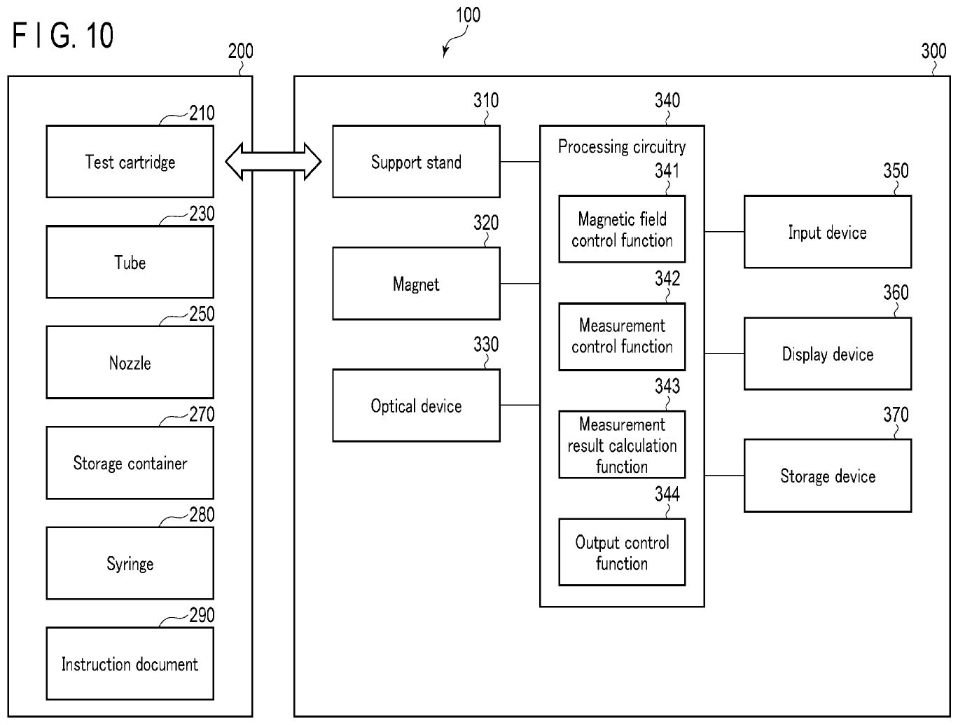

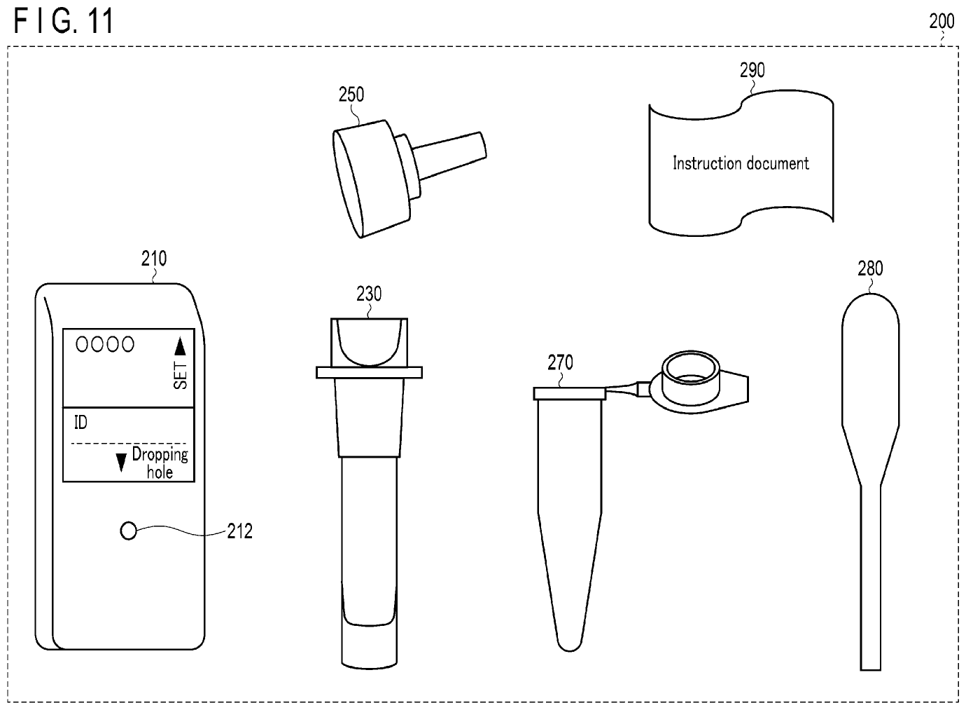

FIG. 10 is a block diagram showing an example of the configuration of an optical measurement system 100 according to the second embodiment. FIG. 11 is a view showing the outer appearance of a test kit 200 according to the second embodiment. As shown in FIGS. 10 and 11, the test kit 200 according to the second embodiment includes a test cartridge 210, a tube 230, a nozzle 250, a storage container 270, a syringe 280, and an instruction document 290.

The storage container 270 is a container for storing a specimen solution containing a specimen, a specimen treatment solution, and magnetic particles. The storage container 270 is a container separated from an optical measurement apparatus 300. A container used as the storage container 270 is not specifically limited as long as it can store the specimen solution. However, a container with a cover is desirable in order to avoid contamination by the outside air. Furthermore, the container desirably has a tapered shape that is tapered from an opening to a tip portion so that the magnetic particles can spontaneously precipitate in the storage container 270 even if a small amount of the specimen solution is stored. As a container that satisfies these conditions, for example, a microtube is appropriate.

The syringe 280 is a tool for sucking a mixed solution stored in the storage container 270 and discharging it to the test cartridge 210. The syringe 280 is not an essential component of the test kit 200. If, for example, it is assumed that an operator uses another additional syringe, it is unnecessary to package the syringe 280 in the test kit 200.

The instruction document 290 is a document in which a sentence that instructs to store the specimen solution for a standby time before introducing the specimen solution to the test cartridge 210 is printed. In the instruction document 290, a URL code with which the sentence can be accessed may be printed, instead of the sentence. As the code, a one- or two-dimensional code is suitable. Typically, the instruction document 290 is a manual of the test kit 200 or the optical measurement apparatus 300. As contents of the manual, the above sentence is described. Note that the instruction document 290 is not an essential component of the test kit 200. The instruction document 290 need not be packaged in the test kit 200 in accordance with the convenience of a distributer or a manufacturer. As an example, in a case where a plurality of test kits 200 are ordered at once, if the same operator receives an additional order and prepares the test kit 200, it is unnecessary to package the instruction document 290.

The processing procedure of optical measurement by the optical measurement system 100 according to the second embodiment will be described next.

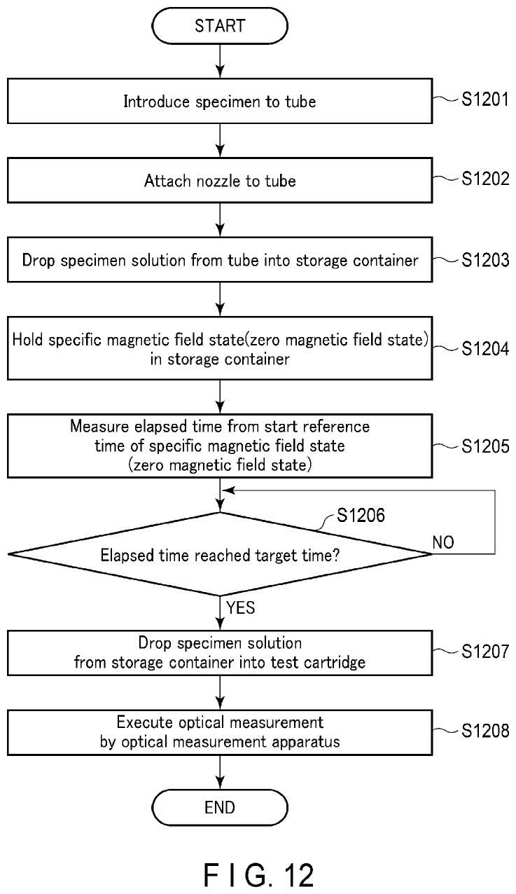

FIG. 12 is a flowchart illustrating the general processing procedure of optical measurement by the optical measurement system 100 according to the second embodiment. In the following description of the second embodiment, assume that a measurement target substance is an antigen, and each of the first substance and the second substance is an antibody. Assume also that the second magnetic particle whose core is not made of a magnetic material is used as a magnetic particle. Note that steps S1201 and S1202 are the same as steps S601 and S602 according to the first embodiment and a description thereof will be omitted.

After step S1202 is performed, the operator drops the specimen solution from the tube 230 into the storage container 270 (step S1203). At this time, if a suspended solution in the tube 230 passes through the filter of the nozzle 250, the magnetic particles immobilized on the filter are eluted to the suspended solution. This introduces the specimen solution as a mixed solution of a specimen, a specimen treatment solution, and the second magnetic particles to the storage container 270.

After step S1203 is performed, the operator holds the specific magnetic field state (zero magnetic field state) in the storage container 270 (step S1204). In step S1204, the operator leaves the storage container 270, to which the specimen solution has been introduced, to stand. While the storage container 270 is left to stand, the magnetic particles in the storage container 270 spontaneously precipitate toward the bottom surface of the storage container 270. Thus, similar to the first embodiment, it is possible to hold the specimen solution in the zero magnetic field state in the storage container 270. That is, since the magnetic particles spontaneously precipitate in the storage container 270 by holding the zero magnetic field state, it is possible to sufficiently ensure a time during which the magnetic particles react with the measurement target substance.

After step S1204 is performed, the operator measures the elapsed time from the start reference time of the specific magnetic field state (zero magnetic field state) (step S1205). The start reference time can be set to arbitrary time such as a point of time at which the specimen solution is introduced to the storage container 270. The method of measuring the elapsed time is not specifically limited, and the elapsed time need only be measured by a time measurement device such as a stopwatch.

After step S1205 is performed, the operator determines whether the elapsed time has reached a target time (standby time) (step S1206). The standby time according to the second embodiment may be set to an arbitrary value without any restriction by the use of the optical measurement apparatus 300, unlike the first embodiment. That is, the standby time may be set to 80 sec, 120 sec, 5 min, 30 min, 60 min, 70 min, 90 min, or any other time. The standby time is qualitatively set to a time longer than an empirical time taken for the second magnetic particles to reach the bottom surface of the storage container 270 from the time at which the second magnetic particles are injected to the storage container 270. The standby time is set to 5 min or longer, and preferably 5 to min.

If it is determined that the elapsed time has reached the target time (standby time) (YES in step S1206), the operator drops the test solution from the storage container 270 into the test cartridge 210 (step S1207). Note that before step S1207, the operator attaches the test cartridge 210 to a support stand 310 of the optical measurement apparatus 300. The operator drops the test solution into a dropping hole 212 of the test cartridge 210 attached to the support stand 310.

After step S1207 is performed, the optical measurement apparatus 300 executes optical measurement for the specimen (step S1208). The optical measurement apparatus 300 executes optical measurement by receiving a measurement start trigger. The measurement start trigger can arbitrarily be set. As an example, the closing of the cover of the optical measurement apparatus 300, a lapse of a preset time after the cover is closed, or the pressing of a measurement start button by the operator is set as the trigger.

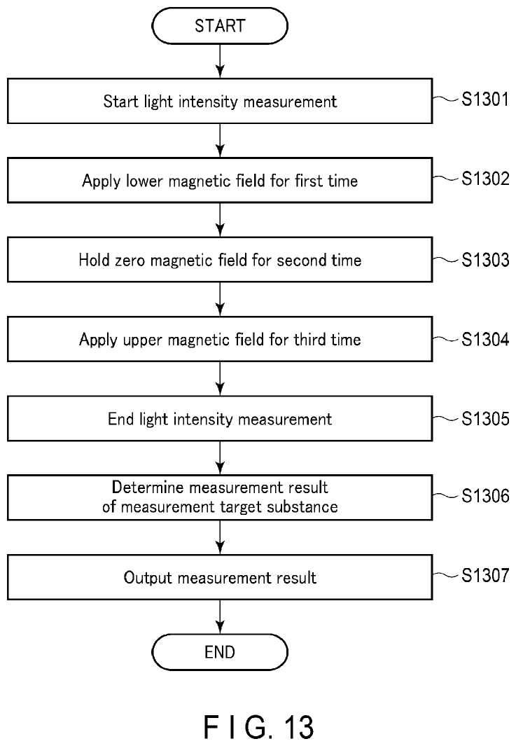

FIG. 13 is a flowchart illustrating the processing procedure of optical measurement executed by the optical measurement apparatus 300 in step S1208 of FIG. 12. FIG. 14 is a timing chart showing a time-series change of a light detection signal according to the second embodiment. In the timing chart of FIG. 14, the ordinate represents the signal strength [AU] of the light detection signal, and the abscissa represents the time [sec]. In FIG. 14, a thick line represents the time-series signal strength of the specimen containing the measurement target substance, and a thin line represents the time-series signal strength of the specimen containing no measurement target substance. Note that the principle of the behavior of the signal strength of the light detection signal according to the second embodiment is the same as in the first embodiment.

As shown in FIG. 13, a processing circuitry 340 of the optical measurement apparatus 300 starts light intensity measurement by a measurement control function 342 (step S1301). The processing contents of the light intensity measurement are the same as in step S701 according to the first embodiment. As shown in FIG. 14, as the time elapses after step S1301, the signal strength of the light detection signal increases.

After step S1301 is performed, the processing circuitry 340 applies a lower magnetic field for the first time by a magnetic field control function 341 (step S1302). The first time according to the second embodiment is equal to the total time of the first time and the target time according to the first embodiment. A method of applying the lower magnetic field in step S1302 is the same as in step S705.

After step S1302 is performed, the processing circuitry 340 holds the zero magnetic field for the second time by the magnetic field control function 341 (step S1303). The second time according to the second embodiment is equal to the second time according to the first embodiment. A method of holding the zero magnetic field in step S1303 is the same as in step S706.

After step S1303 is performed, the processing circuitry 340 applies an upper magnetic field for the third time by the magnetic field control function 341 (step S1304). The third time according to the second embodiment is equal to the third time according to the first embodiment. A method of applying the upper magnetic field in step S1304 is the same as in step S707.

After step S1304 is performed, the processing circuitry 340 ends the light intensity measurement by the measurement control function 342 (step S1305). After step S1305 is performed, the processing circuitry 340 outputs a measurement result calculated in step S1304 by an output control function 344 (step S1306). Steps S1305 and S1306 are the same as steps S709 and S710.

Then, the optical measurement by the optical measurement apparatus 300 ends.

As described above, according to the second embodiment, the optical measurement method by the optical measurement apparatus 300 includes no step of holding the specific magnetic field state. According to the second embodiment, before introducing the specimen solution to the test cartridge 210, the specimen solution containing the magnetic particles and the specimen is held in the storage container 270 in the zero magnetic field state. This holding step makes it possible to sufficiently ensure the time during which the magnetic particles react with the antigen outside the optical measurement apparatus 300, thereby improving the reaction efficiency of the antigen-antibody reaction, and also improving the sensitivity characteristic. Furthermore, according to the second embodiment, since the optical measurement apparatus 300 is not occupied for the holding step, it is possible to improve the throughput of optical measurement including the holding step.

Note that in the above-described embodiment, the storage container 270 has been described as an example of a storage space for storing the specimen solution outside the translucent substrate 214. However, this embodiment is not limited to this. For example, the storage space may be provided in a channel that is provided in the test cartridge 210 and connects the dropping hole 212 and a reaction tank 213. As another example, the storage space may be provided in the tube 230 or the nozzle 250.

### Example 2

Optical measurement was executed by the following procedure using the optical measurement system 100 according to the second embodiment. A nozzle with a SARS-CoV-2 antibody reagent, a sensor cartridge, and a specimen treatment solution were used as the test kit 200, and a protein analysis apparatus “Rapiim Eye 10” (Canon Medical Systems Corporation) was used as the optical measurement apparatus 300.

<Method>

1. Preparation of Specimen Solution

A specimen for management (antigen concentration: 125 pg/mL) was added to 350 μL of a specimen treatment solution in a tube, and the nozzle was attached to the tube. As the specimen for management, a recombinant SARS-CoV-2 antigen (KANTO CHEMICAL CO., INC.) was used. The specimen for management was diluted using 9.6 mM PBS (1% BSA, 0.1% Triton-X added) as an antigen dilution buffer.

2. Static Reaction

Six drops of the specimen solution prepared in 1 were vertically dropped into a microtube from the nozzle. After dropping, the microtube was left to stand for 1, 3, or 5 min, and was subjected to a static reaction.

3. Measurement

After each static reaction, the specimen solution was dropped into the test cartridge, and antigen sensitivity measurement was performed by the following procedure. As control, the specimen solution prepared in 1 was dropped into the test cartridge without being subjected to a static reaction, and antigen sensitivity measurement was performed by the following procedure. (i) The optical measurement apparatus (Rapiim Eye 10) was started. (ii) A cartridge for a coronavirus test was set in the optical measurement apparatus, a sample was sucked from the microtube by a syringe, all of the sample was dropped into the dropping hole of the cartridge, and the slide cover was closed, thereby starting measurement. In this measurement, “the standard sequence of the current product” described in Example 1 was executed.

<Result>

The measurement data of the time-series change of the signal strength was saved, and the sensitivity value (S value) was calculated by equation (1) above. A positive/negative result was determined by comparing the S value with a threshold. The determination threshold was set to attenuation index determination: 12.8.

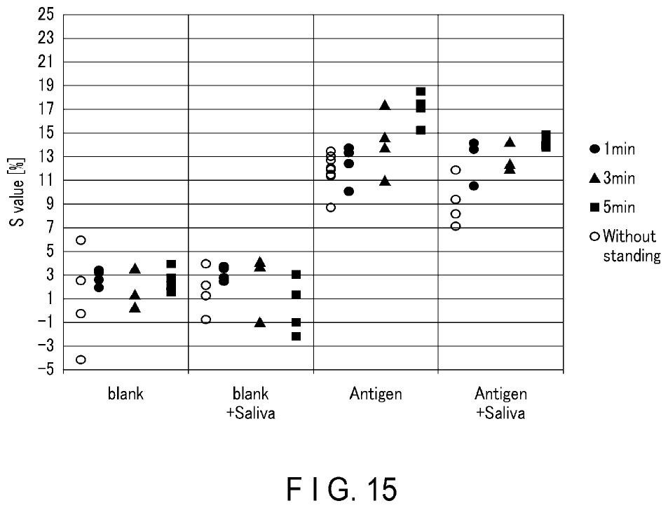

FIG. 15 is a graph showing a measurement result according to Example 2. Referring to FIG. 15, “antigen” represents a case where the specimen for management was added to the specimen treatment solution, “blank” represents a case where nothing was added to the specimen treatment solution, “blank+saliva” represents a case where the saliva of an able-bodied person was added to the specimen treatment solution, and “antigen+saliva” represents a case where the specimen for management and the saliva of an able-bodied person were added to the specimen treatment solution. In FIG. 15, for each of the cases of “blank”, “blank+saliva”, “antigen”, and “antigen+saliva”, the sensitivity values (S values) in a case where no static reaction was performed, a case where a static reaction was performed for 1 min, a case where a static reaction was performed for 3 min, and a case where a static reaction was performed for 5 min are plotted.

It is apparent from the results shown in FIG. 15 that it is possible to improve sensitivity by performing a static reaction before antigen sensitivity measurement. The same experiment was executed by performing a static reaction for 5 min, 30 min, 60 min, 70 min, and 90 min, the influence of the time of the static reaction on the sensitivity was checked. As a result, the sensitivity value in a case where the static reaction was performed for 5 min increased, as compared with control (no static reaction) but the sensitivity value in a case where the static reaction was performed for 30 min or more was almost equal to the sensitivity value in a case where the static reaction was performed for 5 min. Based on the these results, the time of the static reaction can be set to, for example, 5 min or longer, and preferably 5 to 60 min.

### SUMMARY

The optical measurement method according to each of the above-described various embodiments is an optical measurement method using the optical measurement system 100. The optical measurement system 100 includes the translucent substrate 214 which is translucent and on which the first substance be capable of specifically bonded to the measurement target substance is immobilized, the magnet 320 that applies a magnetic field for moving the magnetic particles bonded to the second substance be capable of specifically bonded to the measurement target substance, and the optical device 330 that emits light to the translucent substrate 214 and detects light having propagated through the translucent substrate 214 and exited from the translucent substrate 214. The optical measurement method includes the holding step (S702, S1204), the measurement step (S703, S1205), and the first application step (S705, S1302). In the holding step, the specimen solution containing the specimen and the magnetic particles is held in the specific magnetic field state, in which a magnetic field not lower than the lower magnetic field for moving the magnetic particles closer to the translucent substrate 214 is not applied, in the translucent substrate 214 or the storage unit different from the translucent substrate 214. In the measurement step, the elapsed time from the start reference time of the specific magnetic field state is measured. In the first application step, after the elapsed time reaches the predetermined time, the lower magnetic field is applied, for the first time, by the magnet 320, to the specimen solution introduced to the translucent substrate 214.

The optical measurement system 100 according to each of the above-described various embodiments includes the first substance, the magnetic particles, the translucent substrate 214, the magnet 320, the optical device 330, and the processing circuitry 340. The first substance is capable of specifically bonded to the measurement target substance. The magnetic particles are bonded to the second substance be capable of specifically bonded to the measurement target substance. The first substance is immobilized on the translucent substrate 214, the translucent substrate 214 is translucent, and the specimen solution containing the specimen and the magnetic particles is introduced to the translucent substrate 214. The magnet 320 applies a magnetic field for moving the magnetic particles. The optical device 330 emits light to the translucent substrate 214 and detects light having propagated through the translucent substrate 214 and exited from the translucent substrate 214. The processing circuitry 340 holds the specimen solution in the specific magnetic field state in which a magnetic field not lower than the lower magnetic field for moving the magnetic particles closer to the translucent substrate 214 is not applied. The processing circuitry 340 measures the elapsed time from the start reference time of the specific magnetic field state. After the elapsed time reaches the predetermined time, the processing circuitry 340 applies, for the first time, by the magnet 320, the lower magnetic field to the specimen solution introduced to the translucent substrate 214.

The optical measurement system 100 according to each of the above-described various embodiments includes the first substance, the magnetic particles, the translucent substrate 214, the magnet 320, the optical device 330, and the storage container 270. The first substance is capable of specifically bonded to the measurement target substance. The magnetic particles are bonded to the second substance be capable of specifically bonded to the measurement target substance. The first substance is immobilized on the translucent substrate 214, the translucent substrate 214 is translucent, and the specimen solution containing the specimen and the magnetic particles is introduced to the translucent substrate 214. The magnet 320 applies a magnetic field for moving the magnetic particles. The optical device 330 emits light to the translucent substrate 214 and detects light having propagated through the translucent substrate 214 and exited from the translucent substrate 214. The storage container 270 is a container for storing the specimen solution before introducing it to the translucent substrate 214.

The test kit 200 according to each of the above-described various embodiments includes the test cartridge 210, the tube 230, the nozzle 250, and the storage container 270. The test cartridge 210 includes the translucent substrate 214 on which the first substance that is specifically bonded to the measurement target substance is immobilized. The tube 230 is a container containing the specimen treatment solution. The nozzle 250 is a nozzle which is attached to the tube 230 and to which the magnetic particles bonded to the second substance that is specifically bonded to the measurement target substance adhere. The storage container 270 is a container for storing the specimen solution containing the specimen, the specimen treatment solution, and the magnetic particles before introducing the specimen solution to the translucent substrate 214.

According to the above-described various embodiments, it is possible to provide a time (standby time) during which the specimen solution is held in the specific magnetic field state, before the application of the lower magnetic field. Therefore, it is possible to sufficiently ensure the time during which the magnetic particles react with the measurement target substance. Thus, even if a small amount of the measurement target substance is contained in the specimen, it is possible to improve the reaction efficiency of the magnetic particles and the measurement target substance, as compared with a case where the lower magnetic field is immediately applied without providing the standby time. By providing the step of holding the test solution in the specific magnetic field state, the probability of physical dissociation between the magnetic particles and the measurement target substance decreases, as compared with a case where the step of stirring the specimen solution is provided, and it is thus possible to improve the reaction efficiency of the magnetic particles and the measurement target substance.

According to at least one of the above-described embodiments, it is possible to further improve sensitivity to a smaller amount of the measurement target substance.

The term “processor” used in the above description means, for example, a circuit such as a CPU, a GPU, an Application Specific Integrated Circuit (ASIC), or a programmable logic device (for example, a Simple Programmable Logic Device (SPLD), a Complex Programmable Logic Device (CPLD), or a Field Programmable Gate Array (FPGA)). The processor implements functions by reading out programs stored in a storage circuit and executing them. Note that instead of storing the programs in the storage circuit, the programs may directly be incorporated in the circuit of the processor. In this case, the processor reads out the programs incorporated in the circuit and executes them, thereby the functions. On the other hand, if the processor is, for example, an ASIC, the function is directly be incorporated as a logic circuit in the circuit of the processor instead of storing the program in the storage circuit. Note that ach processor according to each embodiment need not necessarily be configured as a single circuit for each processor. Instead, one processor may be formed by combining a plurality of independent circuits to implement the functions. Furthermore, the plurality of components in FIGS. 1 and 10 may be integrated to one processor, thereby the functions.

Several embodiments have been described above. However, these embodiments are merely examples and are not intended to limit the scope of the present invention. These embodiments can be executed in various other forms, and various omissions, replacements, changes, and embodiments can be combined without departing from the scope of the present invention. These embodiments and modifications are incorporated in the scope of the present invention, and are also incorporated in the invention described in the claims and their equivalents.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An optical measurement method using an optical measurement system including
a translucent substrate on which a first substance capable of being specifically bonded to a measurement target substance is immobilized,
a magnet configured to apply a magnetic field for moving magnetic particles to which a second substance capable of being specifically bonded to the measurement target substance is bonded, and
an optical device configured to emit light to the substrate and detect light having propagated through the substrate and exited from the substrate,
the method comprising:
a holding step of holding a specimen solution containing a specimen and the magnetic particles in a specific magnetic field state, in which a magnetic field not lower than a first magnetic field for moving the magnetic particles closer to the substrate is not applied, on the substrate or in a storage container different from the substrate;
a measurement step of measuring an elapsed time from a start reference time of the specific magnetic field state; and
a first application step of applying, for a first application time, by the magnet, the first magnetic field to the specimen solution introduced to the substrate, after the elapsed time reaches a predetermined time.

2. The method according to claim 1, wherein
the substrate includes an optical waveguide with a sensing area on which the first substance is immobilized, and a reaction tank that forms a space including the sensing area as a bottom surface, and
in the holding step, the specimen solution is held in the reaction tank in the specific magnetic field state.

3. The method according to claim 2, wherein in the holding step, no magnetic field is applied by the magnet in the specific magnetic field state.

4. The method according to claim 2, wherein in the holding step, a magnetic field lower than the first magnetic field and having the same direction as a direction of the first magnetic field is applied by the magnet in the specific magnetic field state.

5. The method according to claim 2, wherein in the holding step, a multistage magnetic field lower than the first magnetic field is time-divisionally applied by the magnet in the specific magnetic field state.

6. The method according to claim 1, wherein
the storage container is a container separated from the substrate, and
in the holding step, the specimen solution is held in the storage container.

7. The method according to claim 6, further comprising a dropping step of dropping the specimen solution from the storage container into the substrate after the elapsed time reaches the predetermined time,
wherein in the first application step, the first magnetic field is applied, for the first application time, by the magnet, to the specimen solution dropped into the substrate.

8. The method according to claim 6, further comprising a second application step of applying, for a second application time, by the magnet, a second magnetic field for moving the magnetic particles away from the substrate after a lapse of the first application time.

9. The method according to claim 8, further comprising a non-application step of applying no magnetic field by the magnet between the application of the first magnetic field and the application of the second magnetic field.

10. The method according to claim 1, wherein the predetermined time is a time longer than the first application time.

11. The method according to claim 10, wherein the first application time is set based on a time taken for the magnetic particles introduced to the substrate to reach a surface of the substrate on which the first substance is immobilized under the application of the first magnetic field.

12. The method according to claim 11, wherein the magnetic particles are first magnetic particles each including a first core having an aggregate of magnetic materials and a hydrophilic layer that covers the first core.

13. The method according to claim 12, wherein a total time of the predetermined time and the first application time is set based on a time taken for second magnetic particles each including a second core made of a polymer and a magnetic layer that covers the second core to reach the surface under the application of the first magnetic field.

14. The method according to claim 1, wherein the start reference time is set to one of a time at which a cover of an optical measurement apparatus including the magnet and the optical device is closed, a time at which a preset time elapses after the time, and a time at which a start instruction is issued by an operator.

15. An optical measurement system comprising:
a first substance configured to be specifically bonded to a measurement target substance;
magnetic particles to which a second substance capable of being specifically bonded to the measurement target substance is bonded;
a substrate on which the first substance is immobilized, which is translucent, and to which a specimen solution containing a specimen and the magnetic particles is introduced;
a magnet configured to apply a magnetic field for moving the magnetic particles; and
an optical device configured to emit light to the substrate and detect light having propagated through the substrate and exited from the substrate,
the system further comprising:
a holder configured to hold the specimen solution in a specific magnetic field state in which a magnetic field higher than a first magnetic field for moving the magnetic particles closer to the substrate is not applied; and
a measurement circuit configured to measure an elapsed time from a start reference time of the specific magnetic field state,
wherein the magnet applies, for a first application time, the first magnetic field to the specimen solution introduced to the substrate after the elapsed time reaches a predetermined time.

16. A test kit comprising:
a substrate on which a first substance capable of being specifically bonded to a measurement target substrate is immobilized;
a first container configured to contain a specimen treatment solution;
a nozzle configured to be attached to the first container and to which magnetic particles bonded to a second substance capable of being specifically bonded to the measurement target substance adhere; and
a second container configured to store a specimen solution containing a specimen, the specimen treatment solution, and the magnetic particles before introducing the specimen solution to the substrate.

17. The kit according to claim 16, further comprising an instruction document on which one of a sentence that instructs to store the specimen solution for a predetermined time before introducing the specimen solution to the substrate and a URL code with which the sentence can be accessed is printed.

18. An optical measurement system comprising:
a test kit defined in claim 16;
a magnet configured to apply a magnetic field for moving magnetic particles; and
an optical device configured to cause light to enter the substrate and detect light having propagated through a substrate and exited from the substrate.

19. The system according to claim 18, wherein
the magnet and the optical device are included in an optical measurement apparatus, and
a second container is a container separated from the optical measurement apparatus.
